(19) United States
(12) Patent Application Publication
(10) Pub. No.: US 2009/0149893 A1
(43) Pub. Date: Jun. 11, 2009

(54) Cancellous Bone Implant for Cartilage Repair

(76) Inventors: Eric J. Semler, Piscataway, NJ (US); Alex B. Callahan, Perth Amboy, NJ (US); Katherine G. Truncale, Hillsborough, NJ (US); Roman Shikhanovich, Edison, NJ (US)

Correspondence Address: GREENBERG TRAURIG, LLP, 200 PARK AVE., P.O. BOX 677, FLORHAM PARK, NJ, 07932, US

(21) Appl. No.: 12/328,306

(22) Filed: Dec. 4, 2008

Related U.S. Application Data
(60) Provisional application No. 60/996,800, filed on Dec. 5, 2007.

(51) Int. Cl. A61F 5/00; A61F 2/02; A61F 2/08

(52) U.S. Cl. 606/86 R; 623/23.72; 623/14.13

(57) ABSTRACT

The invention is directed toward a cartilage repair assembly comprising a shaped allograft construct comprising a cylindrical mineralized cancellous bone base member and a demineralized cancellous bone cap member having a cylindrical top portion and a stem extending from the top portion mounted to the bone base member. The base member has a central bore and a transverse bore which intersect the central bore and the cap member stem has a through going bore which is aligned with the base member transverse bore when the stem is mounted in the central bore to receive a pin member. Milled cartilage particles having a size ranging from 10 to 212 microns are mixed with a biocompatible carrier and a cartilage growth factor, with the mixture being infused in the cap member to generate cartilage growth.

### RELATED APPLICATION

This application claims priority to U.S. Provisional Patent Application No. 60/996,800 filed Dec. 5, 2007, which is incorporated by reference herein in its entirety.

### STATEMENT REGARDING FEDERALLY SPONSORED RESEARCH OR DEVELOPMENT

Not applicable.

### REFERENCE TO SEQUENCE LISTING, A TABLE OR A COMPUTER PROGRAM LISTING COMPACT DISC APPENDIX

None.

### BACKGROUND OF THE INVENTION

1. Field of Invention

The present invention is generally directed toward an allograft cartilage repair implant and is more specifically directed toward a two piece allograft cancellous bone implant having a mineralized cancellous bone base member defining a central blind bore and a bore transverse to the central bore intersecting the central bore and a demineralized cancellous cap member mounted to the base member. The cap member has a cylindrical top section and a stem extending from the top section which has a transverse bore cut therethrough and is placed in the central bore of the base member. A pin is mounted in the transverse bore of the base member through the stem transverse bore. In an alternate embodiment the cap member defines a central blind bore with a bone transverse to the central bore intersecting the central bore. The base member has a cylindrical bottom section and a stem extending from the bottom section which has a transverse bore cut therethrough which is placed in the central bore of the cap member to receive a pin. The implant is shaped for an interference fit implantation in a bore cut in a shoulder, knee, hip, or ankle joint to remove a cartilage defect area.

2. Description of the Prior Art

Articular cartilage injury and degeneration present medical problems to the general population which is constantly addressed by orthopedic surgeons. Every year in the United States, over 500,000 arthroplastic or joint repair procedures are performed. These include approximately 125,000 total hip and 150,000 total knee arthroplasties and over 41,000 open arthroscopic procedures to repair cartilaginous defects of the knee.

In the knee joint, the articular cartilage tissue forms a lining which faces the joint cavity on one side and is linked to the subchondral bone plate by a narrow layer of calcified cartilage tissue on the other side (see FIG. 1). Articular cartilage (hyaline cartilage) consists primarily of extracellular matrix with a sparse population of chondrocytes distributed throughout the tissue. Articular cartilage is composed of chondrocytes, type II collagen fibril meshwork, proteoglycans and water. Active chondrocytes are unique in that they have a relatively low turnover rate and are sparsely distributed within the surrounding matrix. The collagens give the tissue its form and tensile strength and the interaction of proteoglycans with water give the tissue its stiffness to compression, resilience and durability. The hyaline cartilage provides a low friction bearing surface over the bony parts of the joint. If the lining becomes worn or damaged, resulting in lesions, joint movement may be painful or severely restricted. Whereas damaged bone typically can regenerate successfully, hyaline cartilage regeneration is quite limited because of its limited regenerative and reparative abilities.

Articular cartilage lesions generally do not heal, or heal only partially under certain biological conditions due to the lack of nerves, blood vessels and a lymphatic system. The limited reparative capabilities of hyaline cartilage usually results in the generation of repair tissue that lacks the structure and biomechanical properties of normal cartilage. Generally, the healing of the defect results in a fibrocartilaginous repair tissue that lacks the structure and biomedical properties of hyaline cartilage and degrades over the course of time. Articular cartilage lesions are frequently associated with disability and with symptoms such as joint pain, locking phenomena and reduced or disturbed function. These lesions are difficult to treat because of the distinctive structure and function of hyaline cartilage. Such lesions are believed to progress to severe forms of osteoarthritis. Osteoarthritis is the leading cause of disability and impairment in middle-aged and older individuals, entailing significant economic, social and psychological costs. Each year, osteoarthritis accounts for as many as 39 million physician visits and more than 500,000 hospitalizations. By the year 2020, arthritis is expected to affect almost 60 million persons in the United States and to limit the activity of 11.6 million persons.

There are many current therapeutic methods being used. None of these therapies has resulted in the successful regeneration of hyaline-like tissue that withstands normal joint loading and activity over prolonged periods. Currently, the techniques most widely utilized clinically for cartilage defects and degeneration are not articular cartilage substitution procedures, but rather lavage, arthroscopic debridement, and repair stimulation. The direct transplantation of cells or tissue into a defect and the replacement of the defect with biologic or synthetic substitutions presently accounts for only a small percentage of surgical interventions. The optimum surgical goal is to replace the defects with cartilage-like substitutes so as to provide pain relief, reduce effusions and inflammation, restore function, reduce disability and postpone or alleviate the need for prosthetic replacement.

Lavage and arthroscopic debridement involve irrigation of the joint with solutions of sodium chloride, Ringer or Ringer and lactate. The temporary pain relief is believed to result from removing degenerative cartilage debris, proteolytic enzymes and inflammatory mediators. These techniques provide temporary pain relief, but have little or no potential for further healing.

Repair stimulation is conducted by means of drilling, abrasion arthroplasty or microfracture. Penetration into the subchondral bone induces bleeding and fibrin clot formation which promotes initial repair, however, the tissue formed at the cartilage interface is fibrous in nature and not durable. Pain relief is temporary as the tissue exhibits degeneration, loss of resilience, stiffness and wear characteristics over time.

The periosteum and perichondrium have been shown to contain mesenchymal progenitor cells capable of differentiation and proliferation. They have been used as grafts in both animal and human models to repair articular defects. Few patients over 40 years of age obtain good clinical results, which most likely reflect the decreasing population of osteochondral progenitor cells with increasing age. There have also been problems with adhesion and stability of the grafts, which result in their displacement or loss from the repair site.

Transplantation of cells grown in culture provides another method of introducing a new cell population into chondral and osteochondral defects. CARTICEL® is a commercial process to culture a patient's own cartilage cells for use in the repair of cartilage defects in the femoral condyle marketed by Genzyme Biosurgery in the United States and Europe. The procedure uses arthroscopy to take a biopsy from a healthy, less loaded area of articular cartilage of the patient. Enzymatic digestion of the harvested tissue releases the cells that are sent to a laboratory where they are grown for a period ranging from 2-5 weeks. Once cultivated, the cells are injected during a more open and extensive knee procedure into areas of defective cartilage where it is hoped that they will facilitate the repair of damaged tissue. An autologous periosteal flap with a cambium layer is used to seal the transplanted cells in place and act as a mechanical barrier. Fibrin glue is used to seal the edges of the flap. This technique preserves the subchondral bone plate and has reported a high success rate. Proponents of this procedure report that it produces satisfactory results, including the ability to return to demanding physical activities, in more than 90% of patients and those biopsy specimens of the tissue in the graft sites show hyaline-like cartilage repair. More work is needed to assess the function and durability of the new tissue and determine whether it improves joint function and delays or prevents joint degeneration. As with the perichondrial graft, patient/donor age may compromise the success of this procedure as chondrocyte population decreases with increasing age. Disadvantages to this procedure include the need for two separate surgical procedures, potential damage to surrounding cartilage when the periosteal patch is sutured in place, the requirement of demanding microsurgical techniques, and the expensive cost of the procedure resulting from the cell cultivation which is currently not covered by insurance.

Another procedure known as osteochondral transplantation or mosaicplasty involves excising all injured or unstable tissue from the articular defect and creating cylindrical holes in the base of the defect and underlying bone. These holes are filled with autologous cylindrical plugs of healthy cartilage and bone in a mosaic fashion. The filler osteochondral plugs are harvested from a lower weight-bearing area of lesser importance in the same joint. This technique can be performed as arthroscopic or open procedures. Reports of results of osteochondral plug autografts in a small number of patients indicate that they decrease pain and improve joint function, however, long-term results have not been reported. Factors that can compromise the results include donor site morbidity, effects of joint incongruity on the opposing surface of the donor site, damage to the chondrocytes at the articular margins of the donor and recipient sites during preparation and implantation, and collapse or settling of the graft over time. The limited availability of sites for harvest of osteochondral autografts restricts the use of this approach to treatment of relatively small articular defects and the healing of the chondral portion of the autograft to the adjacent articular cartilage remains a concern.

Transplantation of large allografts of bone and overlying articular cartilage is another treatment option that involves a greater area than is suitable for autologous cylindrical plugs, as well as for a non-contained defect. The advantages of osteochondral allografts are the potential to restore the anatomic contour of the joint, lack of morbidity related to graft harvesting, greater availability than autografts and the ability to prepare allografts in any size to reconstruct large defects. Clinical experience with fresh and frozen osteochondral allografts shows that these grafts can decrease joint pain, and that the osseous portion of an allograft can heal to the host bone and the chondral portion can function as an articular surface. Drawbacks associated with this methodology in the clinical situation include the scarcity of fresh donor material and problems connected with the handling and storage of frozen tissue. Fresh allografts carry the risk of immune response or disease transmission. Musculoskeletal Transplant Foundation (MTF) has preserved fresh allografts in a media that maintains a cell viability of 50% for 35 days for use as implants. Frozen allografts lack cell viability and have shown a decreased amount of proteoglycan content which contribute to deterioration of the tissue.

A number of United States Patents have been specifically directed towards bone plugs which are implanted into a bone defect. Examples of such bone plugs are U.S. Pat. No. 4,950,296 issued Aug. 21, 1990 which discloses a bone graft device comprising a cortical shell having a selected outer shape and a cavity formed therein for receiving a cancellous plug, which is fitted into the cavity in a manner to expose at least one surface; U.S. Pat. No. 6,039,762 issued Mar. 21, 2000 discloses a cylindrical shell with an interior body of deactivated bone material; and U.S. Pat. No. 6,398,811 issued Jun. 4, 2002 directed toward a bone spacer which has a cylindrical cortical bone plug with an internal through-going bore designed to hold a reinforcing member. U.S. Pat. No. 6,383,221 issued May 7, 2002 discloses an intervertebral implant having a substantially cylindrical body with a through-going bore dimensioned to receive bone growth materials.

U.S. Pat. No. 6,379,385 issued Apr. 30, 2002 discloses an implant base body of spongious bone material into which a load carrying support element is embedded. The support element can take the shape of a diagonal cross or a plurality of cylindrical pins. See also, U.S. Pat. No. 6,294,187 issued Sep. 25, 2001 which is directed to a load hearing osteoimplant made of compressed bone particles in the form of a cylinder. The cylinder is provided with a plurality of through-going bores to promote blood flow through the osteoimplant or to hold a demineralized bone and glycerol paste mixture. U.S. Pat. No. 6,096,081 issued Aug. 1, 2000 shows a bone dowel with a cortical end cap or caps at both ends, a brittle cancellous body and a through-going bore.

The use of implants for cartilage defects is much more limited. Aside from the fresh allograft implants and autologous implants, U.S. Pat. No. 6,110,209 issued Nov. 5, 1998 shows the use of an autologous articular cartilage cancellous bone paste to fill arthritic defects. The surgical technique is arthroscopic and includes debriding (shaving away loose or fragmented articular cartilage), followed by morselizing the base of the arthritic defect with an awl until bleeding occurs. An osteochondral graft is then harvested from the inner rim of the intercondylar notch using a trephine. The graft is then morselized in a bone graft crusher, mixing the articular cartilage with the cancellous bone. The paste is then pushed into the defect and secured by the adhesive properties of the bleeding bone. The paste can also be mixed with a cartilage stimulating factor, a plurality of cells, or a biological glue. All patients are kept non-weight bearing for four weeks and used a continuous passive motion machine for six hours each night. Histologic appearance of the biopsies has mainly shown a mixture of fibrocartilage with hyaline cartilage. Concerns associated with this method are harvest site morbidity and availability, similar to the mosaicplasty method and retention of the implant in the prepared cartilage defect space.

U.S. Pat. No. 6,379,367 issued Apr. 30, 2002 discloses a plug with a base membrane, a control plug, and a top membrane which overlies the surface of the cartilage covering the defective area of the joint.

U.S. Pat. No. 7,067,123 issued Jun. 27, 2006 is directed toward cartilage defect filler material comprising cartilage pieces ranging from 0.01 mm to 1.0 mm in size in a biological carrier which can be phosphate buffered saline, hyaluronic acid and its derivatives as well as other carriers together with allogenic chondrocytes including an additive which can be growth factors.

### SUMMARY OF THE INVENTION

A cartilage repair allograft construct implant assembly is formed with a cylindrical mineralized cancellous bone base member and a demineralized cancellous cap member mounted to the base member. The cap member is preferably formed with a cylindrical top portion and a stem extending therefrom. The cap member is infused with a cartilage paste having small cartilage pieces ranging from about 10 to about 212 microns in size, a carrier and a FGF-2 variant growth factor and the stem of the cap member is mounted in a central bore cut in the base member and held in place by a pin inserted into a transverse bore in the base member which is aligned with a transverse bore formed in the cap member stem. An alternative embodiment uses an inverted design. The construct is used for replacing articular cartilage defects and is placed in a bore which has been cut into the patient to remove the lesion defect area. Each allograft construct can support the addition of a variety of chondrogenic stimulating factors including, but not limited to morselized allogeneic cartilage, growth factors (e.g., FGF-2, FGF-5, FGF-7, FGF-9, FGF-11, FGF-21, IGF-1, TGF-β, BMP-2, BMP-7, PDGF, VEGF) and variants thereof.

It is an object of the invention to provide an allograft implant for joints which provides pain relief, restores normal finction and will postpone or alleviate the need for prosthetic replacement.

It is also an object of the invention to provide a cartilage repair implant which is easily placed in a cartilage defect area by the surgeon using a minimally invasive technique.

It is still another object of the invention to provide a cartilage repair allograft implant which has load bearing capabilities.

It is further an object of the invention to provide an allograft implant procedure which is applicable for osteochondral defects.

It is yet another object of the invention to provide a cartilage repair implant which facilitates growth of hyaline cartilage in the cartilage defect area.

It is an additional object of the invention to provide a cancellous construct which is treated with chondrogenic stimulating factors.

These and other objects, advantages, and novel features of the present invention will become apparent when considered with the teachings contained in the detailed disclosure along with the accompanying drawings.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is an anatomical illustration of a knee joint having articular cartilage in which a lesion has formed;

FIG. 2 is an exploded perspective view of a multi-piece cancellous construct produced in accordance with an exemplary embodiment of the present invention;

FIG. 3 is a top perspective view of the multi-piece construct of FIG. 2, as assembled;

FIG. 4 is a cross-sectional view of the multi-piece construct of FIG. 2 which has been placed in a bore of a cartilage defect area in a patient according to a method performed in accordance with the present invention;

FIG. 5 is an exploded perspective view of the multi-piece cancellous construct of FIG. 2 incorporating a pin assembly; and

FIG. 6 is an exploded perspective view of a multi-piece cancellous construct produced in accordance with another embodiment of the present invention.

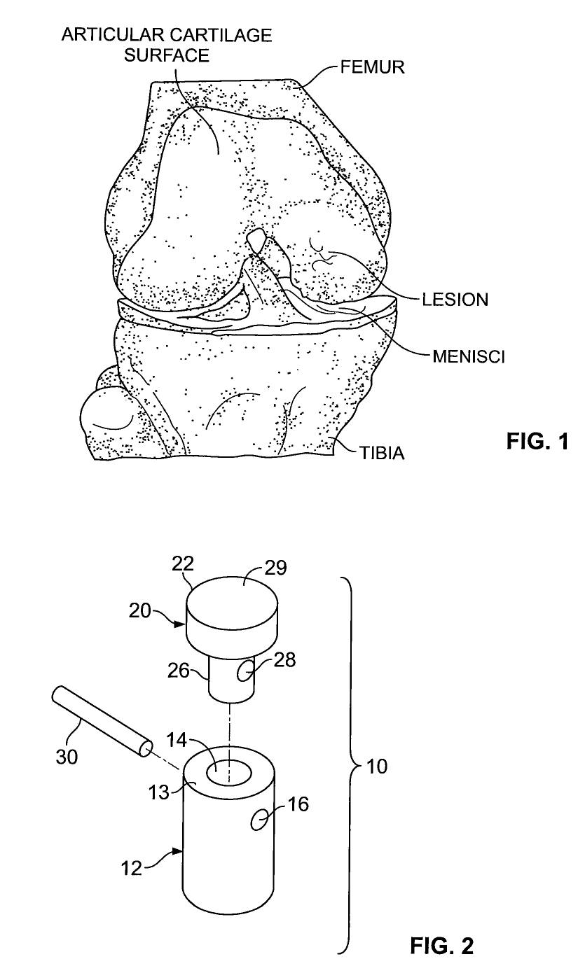

### DESCRIPTION OF THE INVENTION

The term “tissue” is used in the general sense herein to mean any transplantable or implantable tissue, the survivability of which is improved by the methods described herein upon implantation. In particular, the overall durability and longevity of the implant are improved, and host-immune system mediated responses, are substantially eliminated.

The terms “transplant” and “implant” are used interchangeably to refer to tissue, material or cells (xenogeneic or allogeneic) which may be introduced into the body of a patient.

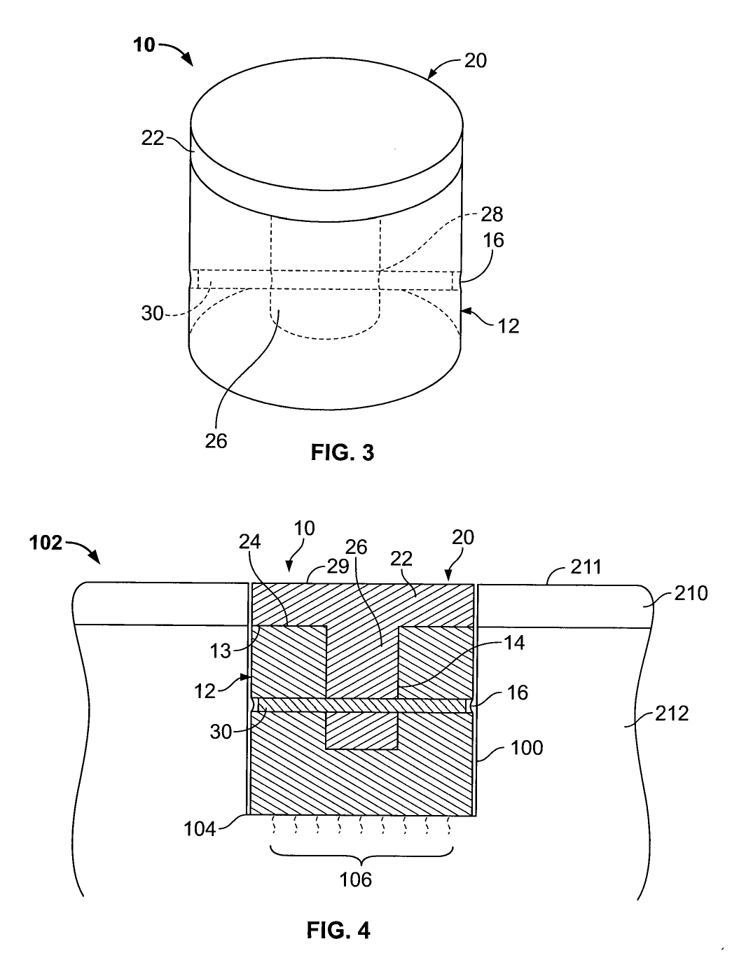

The terms “autologous” and “autograft” refer to tissue or cells which originate with or are derived from the recipient, whereas the terms “allogeneic” and “allograft” refer to cells and tissue which originate with or are derived from a donor of the same species as the recipient. The terms “xenogeneic” and “xenograft” refer to cells or tissue which originate with or are derived from a species other than that of the recipient and the best mode and preferred embodiment is shown in FIGS. 2-5.

The present invention is directed towards a sterile cartilage repair construct constructed of cancellous bone taken from allogenic or xenogenic bone sources.

The construct is preferably derived from dense allograft cancellous bone that may originate from the proximal or distal femur, proximal or distal tibia, proximal humerus, talus, calcaneus, patella, or ilium.

The biphasic design of the scaffold is configured to provide one phase that allows for healing of the cartilage region and another distinct phase that allows for healing of the underlying subchondral bone. The thickness of the top section of the cap member is designed to match or slightly exceed the thickness of the patient's cartilage region. The porous structure of the demineralized cancellous bone in the cap member allows the incorporation and retention of a paste-like matrix of cartilage particles in this region. This cartilage-derived matrix provides the environment and necessary biochemical cues to elicit a healing response from the cells that have infiltrated the scaffold from the surrounding host tissue and bleeding bone. The sponginess of the cap member enables the top surface of the implant to conform to the natural curvature of the joint surface. This conformability of the top of the scaffold permits treatment of large diameter defects without the risk of a proud edge of the implant causing damage to the opposing joint surface during articulation. The base member is similar in structure and composition to the surrounding subchondral bone and is designed to provide mechanical support to the cap member creating a load-bearing scaffold, and also to allow a press-fit into the defect. In addition, the porous nature of the base member enables the bleeding bone to permeate rapidly throughout the scaffold providing the host cells necessary for healing. While the scaffold is preferably constructed with allograft bone, it is also envisioned that the same can be constructed of xenograft bone when the same is properly treated.

Cancellous tissue is first processed into blocks and then milled into the desired shapes for the various components of the invention. In a preferred embodiment, the bicomponent implant assembly 10 is milled using a lathe to form a mineralized cancellous bone base member 12 having a cylindrical shape and a diameter varying between 6-30 mm and a demineralized cap member 20. The base member 12 has a top planar surface 13 and defines a central blind bore 14 cut in and along the central axis of the base member 12. The base member 12 additionally has a through-going transverse bore 16 cut through the diameter which intersects the central bore 14. A demineralized cancellous bone cap member 20 is formed with a cylindrical or disc shaped top section 22 having a thickness similar or greater than the thickness of human articular cartilage, namely about 1.5 mm to about 6.0 mm. The cap member 20 is fully demineralized (<0.5% residual calcium wt/wt) and treated with chemical soaks to be non-osteoinductive. The cap member 20 includes a top section 22 having a planar bottom seating surface 24 which sits on the top planar surface 13 of the base member 12. The top section 22 may have the same diameter as the base member 12 or be of a greater diameter than the base member 12. An integral stem 26 extends perpendicularly outward from the top section 22 and has a diameter smaller than the base member central blind bore 14 so that it fits in the bore 14 of the base member 12. A through-going bore 28 ranging from 1.5 mm to about 3.0 mm in diameter is cut through the mid-section of the stem 26 and when the planar seating surface 24 rests on the top planar surface 13 of the base member 12, the cap member 20 is rotated until the stem bore 28 is aligned with the transverse bore 16 of the base member 12 providing a straight axially aligned combined bore extending through the base member 12 and the stem 26. If desired, the bore 28 and the bore 16 can be angled to provide an angled combined bore through the base member 12 and the stem 26. A cylindrical cancellous bone pin 30 or bone pin assembly 31 is inserted into the axially aligned combined bores 16, 28 to hold the two pieces (i.e., the base member 12 and the cap member 20) in a fixed relationship.

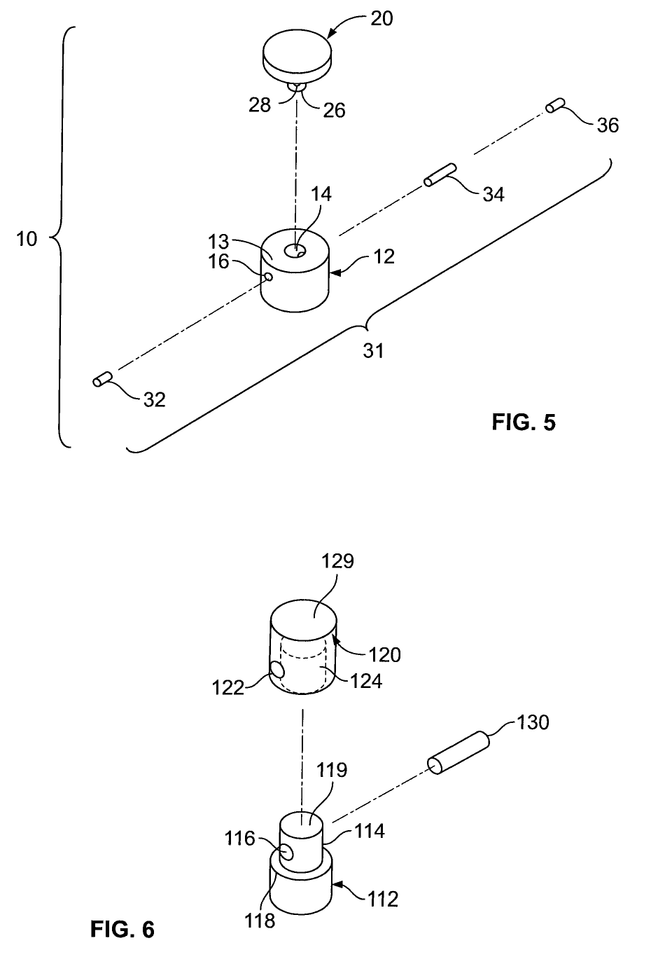

If the implant assembly 10 has a large diameter, multiple pin sections can be used as shown in FIG. 5 to form the bone pin assembly 31. Multiple cancellous pins 32, 34 and 36 are used in sequence to attach the cap member 20 to the base member 12. In this configuration, one pin 32 is inserted into one end of the stem bore 28 through the transverse bore 16, a second longer pin 34 is inserted into the opposite end of the stem bore 28 while the pin 32 is held in place and a third shorter pin 36 is inserted into the stem bore 28 from the same side as the second pin 34. While the bone pin is preferably constructed of cancellous bone or cortical bone, other biocompatible materials such as a ceramic, metal such as surgical steel or a biocompatible polymer can be used.

In an alternate embodiment as shown in FIG. 6 which is an inverted design of the embodiment shown in FIGS. 2-5, a cylindrically shaped base member 112 is stepped at 118 to form a stem 114 having a transverse bore 116 extending through the diameter of the stem 114, with the end surface 119 of the stem 114 being planar to fit against the end surface of bore 124 of the cap member 120. The cap member 120 is cylindrical with a blind bore 124 cut therein to receive the stem 114 and has a transverse bore 122 which intersects the blind bore 124. When the cap member 120 is rotated around the stem 114, the bores 122 and 116 are axially aligned to receive a pin 130 (or a pin assembly as shown in FIG. 5) holding the two pieces of the implant together in a fixed relationship. The top surface 129 of cap member 120 is substantially planar or slightly curved to correspond with the surrounding cartilage area 210 of the patient forming a smooth continuous surface.

The cap member 20/120 is preferably constructed of cancellous bone and is demineralized in dilute acid such as HCL until the bone contains less than 0.5% wt/wt residual calcium. If desired, the cap member 20/120 can be treated so that a section of the stem 26/114 is left mineralized. Subsequently, the resultant demineralized tissue form of the cap member 20/120 is predominantly Type I collagen, which is sponge-like in nature with an elastic quality. Following decalcification, the tissue is further cleaned, brought to a physiological pH level of about 7.0 and treated with chemical soaks of hydrogen peroxide for about 1 hour with ultrasonic so that the cancellous tissue is nonosteoinductive. Alternatively, this inactivation of inherent osteoinductivity of the demineralized cancellous bone may be accomplished via chemical or thermal treatment or by high energy irradiation.

The demineralized cap member 20/120 is infused with a matrix of minced cartilage putty or gel consisting of minced or milled allograft cartilage pieces having a size ranging from about 10 microns to about 212 microns that have been reconstituted in saline. The cartilage particles are preferably allograft cartilage derived from hyaline, fibrous or a combination of hyaline and fibrous cartilage. However, it is also envisioned that autograft or xenograft cartilage may be used. The cartilage particles have been previously lyophilized so that their water content ranges from 0.1% to 8.0% with the cartilage pieces ranging from about 20% to about 40% by weight of the infusion matrix, preferably 22% and mixed with a carrier which can have a composition of one or more of the following: phosphate buffered saline, saline sodium hyaluronate solution (HA) (molecular weight ranging from 7.0×10⁵ to 1.2×10⁶) or other suitable bioabsorbable carrier such as hyaluronic acid and its derivatives, gelatin, collagen, chitosan, alginate, Dextran, carboxymethylcellulose (CMC), hydroxypropyl methylcellulose, or other polymers, the carrier ranging from ranging from about 75% to about 60% by weight. The preferred carrier is phosphate buffered saline at about 22% w/w. Another carrier which can be used is sterile water.

In a most preferred embodiment, morselized cartilage particles having a size less than 212 microns, preferably ranging from about 10 to about 212 microns, are combined with a phosphate buffered saline carrier and a preferred fibroblast growth factor such as FGF-2 variant (FGF-2v) in a dosage of 10-5000 micrograms per cubic cm. This combination is infused into the cap member 20/120. The preferred fibroblast growth factor FGF-2v is described in U.S. Patent Application Publication Number 20050148511 filed Nov. 5, 2004 which is incorporated by reference herein and discloses a variant of FGF-2 having at least one amino acid substitution in the beta 8-beta 9 loop, the variant is characterized in having at least one of the following attributes compared to the corresponding wild type FGF-2: enhanced specificity for one receptor subtype; increased biological activity mediated by at least one receptor subtype with equivalent or reduced activity mediated through another receptor subtype; enhanced affinity to at least one receptor subtype; and increased cell proliferation mediated through one receptor subtype. The demineralized portion will contain approximately 0.1-1.0 g/cc of cartilage paste.

The outer diameter of the assembled implant ranges from between 6-30 mm and its overall height ranges between 8-20 mm.

If desired, the open cancellous structure of the cap member 20 may additionally be loaded with the cartilage pieces and carrier noted above and/or one or more chondrogenic growth factor additives namely recombinant or native or variant growth factors of FGF-2, FGF-5, FGF-7, FGF-9, FGF-11, FGF-21, TGF-β, BMP-2, BMP-4, BMP-7, PDGF, VEGF, and a bioactive peptide such as Nell-1 or TP508. Additional growth factors which can be added are insulin-like growth factor-1 (IGF-1), hepatocyte growth factor and platelet-derived growth factor. Other additives can include human allogenic or autologous chondrocytes, human allogenic cells, human allogenic or autologous bone marrow cells, human allogenic or autologous stem cells, demineralized bone matrix, insulin, insulin-like growth factor-1, interleukin-1 receptor antagonist, hepatocyte growth factor, platelet-derived growth factor, Indian hedgehog, parathyroid hormone-related peptide, viral vectors for DNA delivery, nanoparticles, or platelet-rich plasma. This design enables the fabrication of an implant that possesses a relatively uniform substantially demineralized top section that is distinct from the mineralized base section.

The sterile implant 10 is placed in a defect area bore 100 which has been cut in the lesion area of the bone 102 of a patient with the top surface 29 of the cap member top section 22 being slightly proud, slightly below, or substantially flush with the surface 211 of the original cartilage 210 surrounding the defect bone area remaining at the area being treated (see FIG. 4). The base member 12 and the cap member 20 are force fit into the bore 100 defining the defect area. The diameter of the base member 12 is preferably greater than the diameter of the bore 100 prior to insertion into the bore 100. The implant 10 has a length which can he the same as the depth of the defect bore 100 or more or less than the depth of the bore 100. If the height of the implant 10 is the same as the depth of the bore 100, the base of the implant 10 is supported by the bottom surface of the bore 100 and the top surface 29 of the cap member 20 is substantially level with the surrounding articular cartilage to form a smooth continuous surface and to be load bearing. With such load bearing support the graft surface is not damaged by weight or bearing loads which can cause micromotion interfering with the graft interface producing fibrous tissue interfaces and subchondral cysts.

The invention disclosure also describes the method of treatment of either primary focal lesions in articular cartilage or backfill site defects with the biphasic scaffold. During the treatment of a primary defect, the lesion is first prepared by measuring the defect and coring out the damaged region with a flat-bottom drill. The diameter of the chosen scaffold will be slightly larger than the diameter of the cored defect in order to create a press-fit. The base of the scaffold will be trimmed to match the depth of the defect and the edges of the base may be chamfered to facilitate insertion. The implant will then be inserted in a dry state into the defect site by using a tamp and a mallet or other insertion device. The implant is positioned such that its top surface is either flush, slightly proud, or slightly lower to the surface of the adjacent cartilage. The scaffold is re-hydrated by the bleeding bone from the surrounding host tissue in situ.

During treatment of a backfill defect site, the defect will be created when an osteochondral plug is removed from a non-weight bearing region of the patient's own joint and transferred to a primary defect site. After the backfill site is prepared, the biphasic scaffold will be selected for a press-fit with the defect and will be trimmed to match the depth of the defect. The edges of the base of the scaffold may be chamfered to facilitate insertion. The scaffold will then be implanted in a similar manner for treatment of a primary defect.

In operation, the lesion or defect is removed by cutting a blind bore 100 removing the cartilage 210 having a lesion and the subchondral bone 212 beneath the cartilage defect of the patient. The base 104 of the bore 100 is then micro-fractured 106 to cause bleeding. The implant 10 is then force fit in the bore 100 in an interference fit with the surrounding walls of the bore with the top surface 29 of the cap member section 22 being aligned with the top surface 211 of the cartilage 210 surrounding the implant area of the patient.

If desired, suitable organic glue material can be used to keep the implant components additionally secured together. Suitable organic glue material can be found commercially, such as for example; TISSEEL® or TISSUCOL® (fibrin based adhesive; Immuno AG, Austria), Adhesive Protein (Sigma Chemical, USA), Dow Coming Medical Adhesive B (Dow Corning, USA), fibrinogen thrombin, clastin, collagen, casein, albumin, keratin and the like.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What we claim is:

1. A sterile cartilage repair construct derived from cancellous bone for repair of a defect in articular cartilage comprising a base member of mineralized cancellous bone, a cap member mounted to said base member, means to secure said cap member to said bone member, said cap member being constructed of demineralized cancellous bone, treated to be nonosteoinductive and infused with a composition comprising cartilage particles, a biocompatible carrier and at least one growth factor or bioactive peptide.

2. A sterile cartilage repair construct as claimed in claim 1 wherein said bioactive peptide is taken from a group of bioactive peptides consisting of Nell-1 and TP508.

3. A sterile cartilage repair construct as claimed in claim 1 wherein said base member has a cylindrical shape with a central bore defined therein and a transverse bore intersecting said central bore and said cap member has a cylindrical section and a stem extending from said cylindrical section, said stem defining a through going bore which can be aligned with said base member transverse bore when said stem is mounted in said central bore.

4. A sterile cartilage repair construct as claimed in claim 1 wherein said cap member is constructed of allograft bone.

5. A sterile cartilage repair construct as claimed in claim 1 wherein at least one of said cap member and said base member is constructed of xenograft cancellous bone.

6. A sterile cartilage repair construct as claimed in claim 1 wherein cartilage particles have a size less than 212 microns and form 20-40% w/w of the composition.

7. A sterile cartilage repair construct as claimed in claim 1 wherein cartilage particles have a size ranging from about 10 to about 212 microns.

8. A sterile cartilage repair construct as claimed in claim 1 wherein said cartilage particles are allograft cartilage.

9. A sterile cartilage repair construct as claimed in claim 1 wherein said cartilage particles are autograft cartilage.

10. A sterile cartilage repair construct as claimed in claim 1 wherein said cartilage particles are xenograft cartilage.

11. A sterile cartilage repair construct as claimed in claim 1 wherein said growth factor is FGF-2v.

12. A sterile cartilage repair construct as claimed in claim 1 wherein at least one of said construct members contains one or more of growth factors and variants taken from a group consisting of FGF-2, FGF-5, FGF-7, FGF-9, FGF-11, FGF-21, IGF-1, TGF-β, BMP-2, BMP-4, BMP-7, PDGF, VEGF.

13. A sterile cartilage repair construct as claimed in claim 1 wherein at least one of said construct members contains one or more additives taken from a group consisting of human allogenic or autologous chondrocytes, human allogenic or autologous bone marrow cells and stem cells.

14. A sterile cartilage repair construct as claimed in claim 1 wherein at least one of said construct members contains one or more additives taken from a group consisting of insulin, insulin-like growth factor-1, transforming growth factor-B, interleukin-1 receptor antagonist, hepatocyte growth factor, platelet-derived growth factor, Indian hedgehog and parathyroid hormone-related peptide, bioactive glue, viral vectors for growth factor or DNA delivery, nanoparticles, or platelet-rich plasma.

15. A sterile cartilage repair construct as claimed in claim 1 securing means is at least one pin mounted in said cap member and said base member.

16. A sterile cartilage repair construct as claimed in claim 15 wherein said pin is constructed from a group of materials consisting of mineralized cancellous bone, partially demineralized cortical bone, substantially demineralized cortical bone, cortical bone, ceramic, stainless steel, and polymer.

17. A sterile cartilage repair construct as claimed in claim 16 wherein said pin means is a plurality of cylindrical members.

18. A sterile cartilage repair construct comprising a base member of mineralized cancellous bone, a cap member mounted to said base member, said cap member being constructed of demineralized cancellous bone, and infused with a composition comprising cartilage particles, a biocompatible carrier and a chondrogenic growth factor, said base member has a cylindrical shape with a central bore defined therein and a transverse bore intersecting said central bore, said cap member has a cylindrical section with a stem extending from said cylindrical section, said stem defining a through-going bore which can be aligned with said base member transverse bore when said stem is mounted in said central bore and pin means mounted in said stem bore and said base member transverse bore.

19. A sterile cartilage repair construct comprising a base member of mineralized allograft cancellous bone, a cap member mounted to said base member, said cap member being constructed of demineralized allograft cancellous bone, treated to be non-osteoinductive and infused with a composition comprising allograft cartilage particles having a size ranging from about 10 to about 212 microns, a biocompatible carrier and a chondrogenic growth factor, said base member has a cylindrical shape with a central bore defined therein and a transverse bore intersecting said central bore and said cap member has a cylindrical section with a planar bottom surface and a stem extending from said cylindrical section, said stem defining a through going bore which can be aligned with said base member transverse bore when said stem is mounted in said central bore and a pin mounted through the aligned bores in said base member and said cap member.

20. A sterile cartilage repair construct as claimed in claim 19 wherein said carrier is taken from a group consisting of sterile water, phosphate buffered saline, sodium hyaluronate solution, hyaluronic acid and its derivatives, gelatin, collagen, chitosan, alginate, Dextran, carboxymethylcellulose (CMC), hydroxypropyl methylcellulose.

21. A sterile cartilage repair construct as claimed in claim 19 wherein said allograft cartilage particles are taken from a group consisting of hyaline cartilage, fibrous cartilage and a combination of hyaline and fibrous cartilage.

22. A sterile cartilage repair construct as claimed in claim 19 wherein said fibroblast growth factor FGF-2v is present in an amount of 10-5000 micrograms per cm³.

23. A sterile cartilage repair construct comprising a base member of mineralized allograft cancellous bone, a cap member mounted to said base member, said cap member being constructed of demineralized allograft cancellous bone, treated to be non-osteoinductive and infused with a composition comprising allograft cartilage particles, a biocompatible carrier and a growth factor, said cap member having a cylindrical shape with a central bore defined therein and a transverse bore intersecting said central bore, said base member defining a cylindrical section with a planar bottom surface and a stem extending from said cylindrical section, said stem defining a through going bore which can be aligned with said cap member transverse bore when said base member stem is mounted in said cap member central bore and a pin means mounted through the aligned bores in said base member and said cap member.

24. A process for constructing a sterile cartilage repair construct comprising the steps of:
a. milling a mineralized cancellous bone into a cylindrically shaped base member;
b. demineralizing a cap member adapted to be mounted to the base member;
c. treating the cap member to be non-osteoinductive;
d. mounting the cap member to the base member; and
e. infusing cartilage particles and at least one cartilage growth factor carried in a biocompatible carrier into the cap member.

25. A process as claimed in claim 24 wherein the said cartilage growth factor is FGF-2v.
